(19) United States
(12) Patent Application Publication
(10) Pub. No.: US 2008/0207759 A1
(43) Pub. Date: Aug. 28, 2008

(54) METHOD FOR PROTECTING MITOCHONDRIA

(75) Inventors: Ryuji UENO, Montgomery, MD (US); Sachiko Kuno, Montgomery, MD (US); John Cuppoletti, Cincinnati, OH (US)

Correspondence Address: SUGHRUE MION, PLLC, 2100 PENNSYLVANIA AVENUE, N.W., SUITE 800, WASHINGTON, DC, 20037, US

(73) Assignee: SUCAMPO AG, Zug (CH)

(21) Appl. No.: 12/037,435

(22) Filed: Feb. 26, 2008

Related U.S. Application Data
(60) Provisional application No. 60/903,525, filed on Feb. 27, 2007.

(51) Int. Cl. A61K 31/5575; A61P 43/00

(52) U.S. Cl. 514/573

(57) ABSTRACT

The present invention relates to a method for protecting mitochondria from damage in a mammalian subject, which comprises administering an effective amount of a prostaglandin compound to a subject in need thereof. Also provided is a method for treating mitochondrial dysfunction as well as a condition associated with mitochondrial dysfunction in a mammalian subject, which comprises administering an effective amount of a prostaglandin compound to a subject in need thereof.

### CROSS REFERENCE TO RELATED APPLICATION

This application claims the benefit of U.S. Provisional Application No. 60/903,525 filed Feb. 27, 2007. All contents of the provisional application are herewith incorporated.

### TECHNICAL FIELD

The present invention relates to a method and composition for protecting mitochondria from damage in a mammalian subject.

The present invention also relates to a method and composition for treating a mitochondrial dysfunction in a mammalian subject.

Particularly, the present invention relates to a method and composition for treating a condition associated with mitochondrial dysfunction in a mammalian subject.

### BACKGROUND ART

Mitochondria are subcellular organelles present in all oxygen-utilizing organisms in which energy in the form of adenosine triphosphate (ATP) is generated, and oxygen is reduced to water. Ninety percent of the oxygen taken in is consumed in mitochondria. A substantial byproduct of this ATP generation is potentially toxic oxygen radicals. For example, it is estimated that 1-2% of all reduced oxygen yields superoxide and hydrogen peroxide. Other reactive oxygen species (ROS) formed are singlet oxygen and hydroxyl radical. Under stress conditions in the cell, the ratio of the oxygen radicals can rise to 10% of all consumed oxygen. Mitochondrial membranes are sensitive to lipid peroxidation and depolarization resulting from these ROS. Mitochondrial damage is also a result of exposure to sunlight, which forms ROS as indicated above. Because mitochondria are in cells all over the body and damage to mitochondria is believed to be the cause or an important factor in some diseases, such as brain, nerves, muscles, heart, eyes, kidneys or respiratory problems, a method of protecting mitochondria from such damage, or of repairing such damage, is desired. Cellular damage from burns to the skin and lungs from contact with or exposure to fire and other sources of intense heat is mediated through radical damage. Furthermore, exposure to adverse environmental factors, including industrial air pollutants and petroleum and tobacco combustion products, may contribute to oxidative damage to pulmonary and other tissues of the body. In addition, various therapeutic regimens such as chemotherapeutic drugs and radiation therapy for the treatment of dysproliferative diseases induce significant oxidant-stress-related side effects, such as cardiotoxicity. A lifetime of mitochondrial damage may be a part of the aging process. Agents which protect the mitochondria from such damage will be highly useful, and such agents are strongly desired.

Prostaglandins (hereinafter, referred to as PG(s)) are members of class of organic carboxylic acids, which are contained in tissues or organs of human or other mammals, and exhibit a wide range of physiological activity. PGs found in nature (primary PGs) generally have a prostanoic acid skeleton as shown in the formula (A):

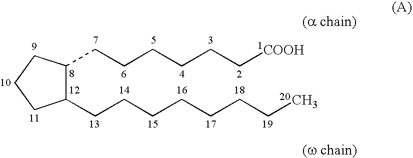

On the other hand, some of synthetic analogues of primary PGs have modified skeletons. The primary PGs are classified into PGAs, PGBs, PGCs, PGDs, PGEs, PGFs, PGGs, PGHs, PGIs and PGJs according to the structure of the five-membered ring moiety, and further classified into the following three types by the number and position of the unsaturated bond at the carbon chain moiety:

Subscript 1: 13,14-unsaturated-15-OH

Subscript 2: 5,6- and 13,14-diunsaturated-15-OH

Subscript 3: 5,6-, 13,14-, and 17,18-triunsaturated-15-OH.

Further, the PGFs are classified, according to the configuration of the hydroxyl group at the 9-position, into α type (the hydroxyl group is of an α-configuration) and β type (the hydroxyl group is of a β-configuration).

PGs are known to have various pharmacological and physiological activities, for example, vasodilatation, inducing of inflammation, platelet aggregation, stimulating uterine muscle, stimulating intestinal muscle, anti-ulcer effect and the like.

Some 15-keto (i.e., having oxo at the 15-position instead of hydroxy)-PGs and 13,14-dihydro (i.e., having single bond between the 13 and 14-position)-15-keto-PGs are known as the substances naturally produced by the action of enzymes during the metabolism of primary PGs.

It is not known how the prostaglandin compound acts on the mitochondria.

### DISCLOSURE OF THE INVENTION

An object of the present invention is to provide a method and composition for protecting mitochondria from damage in a mammalian subject. An additional object of the present invention is to provide a method and composition for treating mitochondrial dysfunction and a condition or disease associated with mitochondrial dysfunction.

Namely, the present invention relates to a method for protecting mitochondria from damage in a mammalian subject, which comprises administering an effective amount of a prostaglandin compound to a subject in need thereof.

The present invention further provides a pharmaceutical composition for protecting mitochondria from damage in a mammalian subject, which comprises an effective amount of a prostaglandin compound.

The present invention still further provides use of a prostaglandin compound for the manufacture of a pharmaceutical composition for protecting mitochondria in a mammalian subject from damage.

The present invention also relates to a method for treating a mitochondrial dysfunction in a mammalian subject, which comprises an effective amount of a prostaglandin compound.

The present invention further provides a pharmaceutical composition for treating a mitochondrial dysfunction in a mammalian subject, which comprises an effective amount of a prostaglandin compound.

The present invention still further provides use of a prostaglandin compound for the manufacture of a pharmaceutical composition for treating a mitochondrial dysfunction in a mammalian subject.

Particularly, the present invention relates to a method for treating a condition associated with mitochondrial dysfunction in a mammalian subject, which comprises administering an effective amount of a prostaglandin compound to a subject in need thereof.

The present invention further provides a pharmaceutical composition for treating a condition associated with mitochondrial dysfunction in a mammalian subject, which comprises an effective amount of a prostaglandin compound.

The present invention still further provides use of a prostaglandin compound for the manufacture of a pharmaceutical composition for treating a condition associated with mitochondrial dysfunction in a mammalian subject.

### BRIEF DESCRIPTION OF THE DRAWINGS

FIG. 1. Protective effect of compound 1 on ET-1 induced loss of mitochondrial membrane potential of human pulmonary artery smooth muscle cells (PASMC).

FIG. 2. Protection by Compound 1 against Endothelin-1 (ET-1) induced loss of mitochondrial membrane potential of human pulmonary artery smooth muscle cells (PASMC).

FIG. 3. Rescue effect of Compound 1 on ET-1 induced loss of mitochondrial membrane potential.

FIG. 4. Cytochrome c translocation from A549 mitochondria incubated for 24 hours in the presence of CSE and/or 100 nM Compound A.

### DETAILED DESCRIPTION OF THE INVENTION

The nomenclature of the prostaglandin compounds used herein is based on the numbering system of the prostanoic acid represented in the above formula (A).

The formula (A) shows a basic skeleton of the C-20 carbon atoms, but the present invention is not limited to those having the same number of carbon atoms. In the formula (A), the numbering of the carbon atoms which constitute the basic skeleton of the PG compounds starts at the carboxylic acid (numbered 1), and carbon atoms in the α-chain are numbered 2 to 7 towards the five-membered ring, those in the ring are 8 to 12, and those in the ω-chain are 13 to 20. When the number of carbon atoms is decreased in the α-chain, the number is deleted in the order starting from position 2; and when the number of carbon atoms is increased in the α-chain, compounds are named as substitution compounds having respective substituents at position 2 in place of the carboxy group (C-1). Similarly, when the number of carbon atoms is decreased in the w-chain, the number is deleted in the order starting from position 20; and when the number of carbon atoms is increased in the ω-chain, the carbon atoms beyond position 20 are named as substituents. Stereochemistry of the compounds is the same as that of the above formula (A) unless otherwise specified.

In general, each of the terms PGD, PGE and PGF represents a PG compound having hydroxy groups at positions 9 and/or 11, but in the present specification, these terms also include those having substituents other than the hydroxy group at positions 9 and/or 11. Such compounds are referred to as 9-dehydroxy-9-substituted-PG compounds or 11-dehydroxy-11-substituted-PG compounds. A PG compound having hydrogen in place of the hydroxy group is simply named as 9- or 11-deoxy-PG compound.

As stated above, the nomenclature of the PG compounds is based on the prostanoic acid skeleton. However, in case the compound has a similar partial structure as a prostaglandin, the abbreviation of “PG” may be used. Thus, a PG compound of which α-chain is extended by two carbon atoms, that is, having 9 carbon atoms in the α-chain is named as 2-decarboxy-2-(2-carboxyethyl)-PG compound. Similarly, a PG compound having 11 carbon atoms in the α-chain is named as 2-decarboxy-2-(4-carboxybutyl)-PG compound. Further, a PG compound of which ω-chain is extended by two carbon atoms, that is, having 10 carbon atoms in the ω-chain is named as 20-ethyl-PG compound. These compounds, however, may also be named according to the IUPAC nomenclatures.

Examples of the analogs (including substituted derivatives) or derivatives include a PG compound of which carboxy group at the end of α-chain is esterified; a compound of which α-chain is extended; physiologically acceptable salt thereof; a compound having a double bond at 2-3 position or a triple bond at position 5-6, a compound having substituent(s) at position 3, 5, 6, 16, 17, 18, 19 and/or 20; and a compound having lower alkyl or a hydroxy (lower) alkyl group at position 9 and/or 11 in place of the hydroxy group.

According to the present invention, preferred substituents at position 3, 17, 18 and/or 19 include alkyl having 1-4 carbon atoms, especially methyl and ethyl. Preferred substituents at position 16 include lower alkyl such as methyl and ethyl, hydroxy, halogen atoms such as chlorine and fluorine, and aryloxy such as trifluoromethylphenoxy. Preferred substituents at position 17 include lower alkyl such as methyl and ethyl, hydroxy, halogen atoms such as chlorine and fluorine, aryloxy such as trifluoromethylphenoxy. Preferred substituents at position 20 include saturated or unsaturated lower alkyl such as C1-4 alkyl, lower alkoxy such as C1-4 alkoxy, and lower alkoxy alkyl such as C1-4 alkoxy-C1-4 alkyl. Preferred substuents at position 5 include halogen atoms such as chlorine and fluorine. Preferred substituents at position 6 include an oxo group forming a carbonyl group. Stereochemistry of PGs having hydroxy, lower alkyl or hydroxy(lower)alkyl substituent at position 9 and/or 11 may be α, β or a mixture thereof.

Further, the above analogs or derivatives may be compounds having an alkoxy, cycloalkyl, cycloalkyloxy, phenoxy or phenyl group at the end of the co-chain where the chain is shorter than the primary PGs.

A prostaglandin compound used in the present invention is represented by the formula (I):

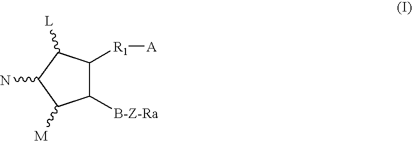

wherein L, M and N are hydrogen, hydroxy, halogen, lower alkyl, hydroxy(lower)alkyl, lower alkanoyloxy or oxo, wherein at least one of L and M is a group other than hydrogen, and the five-membered ring may have at least one double bond;

A is —CH₃, or —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;

Z is

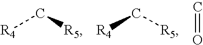

or single bond

wherein R₄ and R₅ are hydrogen, hydroxy, halogen, lower alkyl, lower alkoxy or hydroxy(lower)alkyl, wherein R₄ and R₅ are not hydroxy and lower alkoxy at the same time;

R₁ is a saturated or unsaturated bivalent lower or medium aliphatic hydrocarbon residue, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur; and

Ra is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group.

A preferred compound used in the present invention is represented by the formula (II):

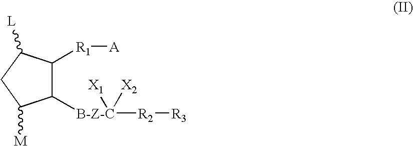

wherein L and M are hydrogen atom, hydroxy, halogen, lower alkyl, hydroxy(lower)alkyl, lower alkanoyloxy or oxo, wherein at least one of L and M is a group other than hydrogen, and the five-membered ring may have one or more double bonds;

A is —CH₃, or —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;

Z is

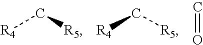

or single bond

wherein R₄ and R₅ are hydrogen, hydroxy, halogen, lower alkyl, lower alkoxy or hydroxy(lower)alkyl, wherein R₄ and R₅ are not hydroxy and lower alkoxy at the same time;

X₁ and X₂ are hydrogen, lower alkyl, or halogen;

R₁ is a saturated or unsaturated bivalent lower or medium aliphatic hydrocarbon residue, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur;

R₂ is a single bond or lower alkylene; and

R₅ is lower alkyl, lower alkoxy, lower alkanoyloxy, cyclo(lower) alkyl, cyclo(lower) alkyloxy, aryl, aryloxy, heterocyclic group or heterocyclic-oxy group.

In the above formula, the term “unsaturated” in the definitions for R₁ and Ra is intended to include at least one or more double bonds and/or triple bonds that are isolatedly, separately or serially present between carbon atoms of the main and/or side chains. According to the usual nomenclature, an unsaturated bond between two serial positions is represented by denoting the lower number of the two positions, and an unsaturated bond between two distal positions is represented by denoting both of the positions.

The term “lower or medium aliphatic hydrocarbon” refers to a straight or branched chain hydrocarbon group having 1 to 14 carbon atoms (for a side chain, 1 to 3 carbon atoms are preferable) and preferably 1 to 10, especially 1 to 8 carbon atoms.

The term “halogen atom” covers fluorine, chlorine, bromine and iodine.

The term “lower” throughout the specification is intended to include a group having 1 to 6 carbon atoms unless otherwise specified.

The term “lower alkyl” refers to a straight or branched chain saturated hydrocarbon group containing 1 to 6 carbon atoms and includes, for example, methyl, ethyl, propyl, isopropyl, butyl, isobutyl, t-butyl, pentyl and hexyl.

The term “lower alkylene” refers to a straight or branched chain bivalent saturated hydrocarbon group containing 1 to 6 carbon atoms and includes, for example, methylene, ethylene, propylene, isopropylene, butylene, isobutylene, t-butylene, pentylene and hexylene.

The term “lower alkoxy” refers to a group of lower alkyl-O—, wherein lower alkyl is as defined above.

The term “hydroxy(lower)alkyl” refers to a lower alkyl as defined above which is substituted with at least one hydroxy group such as hydroxymethyl, 1-hydroxyethyl, 2-hydroxyethyl and 1-methyl-1-hydroxyethyl.

The term “lower alkanoyloxy” refers to a group represented by the formula RCO—O—, wherein RCO— is an acyl group formed by oxidation of a lower alkyl group as defined above, such as acetyl.

The term “cyclo(lower)alkyl” refers to a cyclic group formed by cyclization of a lower alkyl group as defined above but contains three or more carbon atoms, and includes, for example, cyclopropyl, cyclobutyl, cyclopentyl and cyclohexyl.

The term “cyclo(lower)alkyloxy” refers to the group of cyclo(lower)alkyl-O—, wherein cyclo(lower)alkyl is as defined above.

The term “aryl” may include unsubstituted or substituted aromatic hydrocarbon rings (preferably monocyclic groups), for example, phenyl, tolyl, xylyl. Examples of the substituents are halogen atom and halo(lower)alkyl, wherein halogen atom and lower alkyl are as defined above.

The term “aryloxy” refers to a group represented by the formula ArO—, wherein Ar is aryl as defined above.

The term “heterocyclic group” may include mono- to tri-cyclic, preferably monocyclic heterocyclic group which is 5 to 14, preferably 5 to 10 membered ring having optionally substituted carbon atom and 1 to 4, preferably 1 to 3 of 1 or 2 type of hetero atoms selected from nitrogen atom, oxygen atom and sulfur atom. Examples of the heterocyclic group include furyl, thienyl, pyrrolyl, oxazolyl, isoxazolyl, thiazolyl, isothiazolyl, imidazolyl, pyrazolyl, furazanyl, pyranyl, pyridyl, pyridazinyl, pyrimidyl, pyrazinyl, 2-pyrrolinyl, pyrrolidinyl, 2-imidazolinyl, imidazolidinyl, 2-pyrazolinyl, pyrazolidinyl, piperidino, piperazinyl, morpholino, indolyl, benzothienyl, quinolyl, isoquinolyl, purinyl, quinazolinyl, carbazolyl, acridinyl, phenanthridinyl, benzimidazolyl, benzimidazolinyl, benzothiazolyl, phenothiazinyl Examples of the substituent in this case include halogen, and halogen substituted lower alkyl group, wherein halogen atom and lower alkyl group are as described above.

The term “heterocyclic-oxy group” means a group represented by the formula HcO—, wherein Hc is a heterocyclic group as described above.

The term “functional derivative” of A includes salts (preferably pharmaceutically acceptable salts), ethers, esters and amides.

Suitable “pharmaceutically acceptable salts” include conventionally used non-toxic salts, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt (such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino)ethane salt, monomethyl-monoethanolamine salt, procaine salt and caffeine salt), a basic amino acid salt (such as arginine salt and lysine salt), tetraalkyl ammonium salt and the like. These salts may be prepared by a conventional process, for example from the corresponding acid and base or by salt interchange.

Examples of the ethers include alkyl ethers, for example, lower alkyl ethers such as methyl ether, ethyl ether, propyl ether, isopropyl ether, butyl ether, isobutyl ether, t-butyl ether, pentyl ether and 1-cyclopropyl ethyl ether; and medium or higher alkyl ethers such as octyl ether, diethylhexyl ether, lauryl ether and cetyl ether; unsaturated ethers such as oleyl ether and linolenyl ether; lower alkenyl ethers such as vinyl ether, allyl ether; lower alkynyl ethers such as ethynyl ether and propynyl ether; hydroxy(lower)alkyl ethers such as hydroxyethyl ether and hydroxyisopropyl ether; lower alkoxy (lower)alkyl ethers such as methoxymethyl ether and 1-methoxyethyl ether; optionally substituted aryl ethers such as phenyl ether, tosyl ether, t-butylphenyl ether, salicyl ether, 3,4-di-methoxyphenyl ether and benzamidophenyl ether; and aryl(lower)alkyl ethers such as benzyl ether, trityl ether and benzhydryl ether.

Examples of the esters include aliphatic esters, for example, lower alkyl esters such as methyl ester, ethyl ester, propyl ester, isopropyl ester, butyl ester, isobutyl ester, t-butyl ester, pentyl ester and 1-cyclopropylethyl ester; lower alkenyl esters such as vinyl ester and allyl ester; lower alkynyl esters such as ethynyl ester and propynyl ester; hydroxy(lower)alkyl ester such as hydroxyethyl ester; lower alkoxy (lower) alkyl esters such as methoxymethyl ester and 1-methoxyethyl ester; and optionally substituted aryl esters such as, for example, phenyl ester, tolyl ester, t-butylphenyl ester, salicyl ester, 3,4-di-methoxyphenyl ester and benzamidophenyl ester; and aryl(lower)alkyl ester such as benzyl ester, trityl ester and benzhydryl ester.

The amide of A mean a group represented by the formula —CONR′R″, wherein each of R′ and R″ is hydrogen, lower alkyl, aryl, alkyl- or aryl-sulfonyl, lower alkenyl and lower alkynyl, and include for example lower alkyl amides such as methylamide, ethylamide, dimethylamide and diethylamide; arylamides such as anilide and toluidide; and alkyl- or aryl-sulfonylamides such as methylsulfonylamide, ethylsulfonyl-amide and tolylsulfonylamide.

Preferred examples of L and M include hydrogen, hydroxy and oxo, and especially, M is hydroxy and L is oxo which has a 5-membered ring structure of, so called, PGE type.

Preferred example of A is —COOH, its pharmaceutically acceptable salt, ester or amide thereof.

Preferred example of X₁ and X₂ are both being halogen atoms, and more preferably, fluorine atoms, so called 16,16-difluoro type.

Preferred R₁ is a hydrocarbon residue containing 1-10 carbon atoms, preferably 6-10 carbon atoms. Further, at least one carbon atom in the aliphatic hydrocarbon is optionally substituted by oxygen, nitrogen or sulfur.

Examples of R₁ include, for example, the following groups:

—CH₂—CH₂—CH₂—CH₂—CH₂—CH₂—,

—CH₂—CH═CH—CH₂—CH₂—CH₂—,

—CH₂—CH₂—CH₂—CH₂—CH═CH—,

—CH₂—C≡C—CH₂—CH₂—CH₂—,

—CH₂—CH₂—CH₂—CH₂—O—CH₂—,

CH₂—CH═CH—CH₂—O—CH₂—,

—CH₂—C≡C—CH₂—O—CH₂—,

—CH₂—CH₂—CH₂—CH₂—CH₂—CH₂—CH₂—,

—CH₂—CH═CH—CH₂—CH₂—CH₂—CH₂

—CH₂—CH₂—CH₂—CH₂—CH₂—CH═CH—,

—CH₂—C≡C—CH₂—CH₂—CH₂—CH₂—,

—CH₂—CH₂—CH₂—CH₂—CH₂—CH(CH₃)—CH₂,

—CH₂—CH₂—CH₂—CH₂—CH(CH₃)—CH₂—,

—CH₂—CH₂—CH₂—CH₂—CH₂—CH₂—CH₂—CH₂,

—CH₂—CH═CH—CH₂—CH₂—CH₂—CH₂—CH₂—,

—CH₂—CH₂—CH₂—CH₂—CH₂—CH₂—CH═CH—,

—CH₂—C≡C—CH₂—CH₂—CH₂—CH₂—CH₂—, and

—CH₂—CH₂—CH₂—CH₂—CH₂—CH₂—CH(CH₃)—CH₂—.

Preferred Ra is a hydrocarbon containing 1-10 carbon atoms, more preferably, 1-8 carbon atoms. Ra may have one or two side chains having one carbon atom.

Preferable compounds include Ra is substituted by halogen and/or Z is C═O in the formula (I), or one of X1 and X2 is substituted by halogen and/or Z is C═O in the formula (II).

Most preferred embodiment is a prostaglandin compound is 11-deoxy-13,14-dihydro-15-keto-16,16-difluoro-prostaglandin E₁ compound or 13,14-dihydro-15-keto-16,16-difluoro-18-methyl-prostaglandin E₁ compound.

The configuration of the ring and the α- and/or ω chains in the above formula (I) and (II) may be the same as or different from that of the primary PGs. However, the present invention also includes a mixture of a compound having a primary type configuration and a compound of a non-primary type configuration.

In the present invention, the PG compound which is dihydro between 13 and 14, and keto(═O) at 15 position may be in the keto-hemiacetal equilibrium by formation of a hemiacetal between hydroxy at position 11 and keto at position 15.

For example, it has been revealed that when both of X₁ and X₂ are halogen atoms, especially, fluorine atoms, the compound contains a tautomeric isomer, bicyclic compound.

If such tautomeric isomers as above are present, the proportion of both tautomeric isomers varies with the structure of the rest of the molecule or the kind of the substituent present. Sometimes one isomer may predominantly be present in comparison with the other. However, it is to be appreciated that the present invention includes both isomers.

Further, the 15-keto-PG compounds used in the invention include the bicyclic compound and analogs or derivatives thereof.

The bicyclic compound is represented by the formula (III)

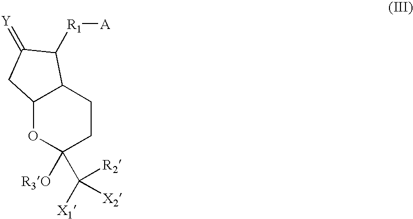

wherein, A is —CH₃, or —CH₂OH, —COCH₂OH, —COOH or a functional derivative thereof;

X₁′ and X₂′ are hydrogen, lower alkyl, or halogen;

Y is

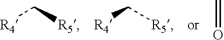

wherein R₄′ and R₅′ are hydrogen, hydroxy, halogen, lower alkyl, lower alkoxy or hydroxy(lower)alkyl, wherein R₄′ and R₅′ are not hydroxy and lower alkoxy at the same time.

R₁ is a saturated or unsaturated divalent lower or medium aliphatic hydrocarbon residue, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur; and

R₂′ is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group.

R₃′ is hydrogen, lower alkyl, cyclo(lower)alkyl, aryl or heterocyclic group.

Furthermore, while the compounds used in the invention may be represented by a formula or name based on keto-type regardless of the presence or absence of the isomers, it is to be noted that such structure or name does not intend to exclude the hemiacetal type compound.

In the present invention, any of isomers such as the individual tautomeric isomers, the mixture thereof, or optical isomers, the mixture thereof, a racemic mixture, and other steric isomers may be used in the same purpose.

Some of the compounds used in the present invention may be prepared by the method disclosed in U.S. Pat. Nos. 5,073,569, 5,166,174, 5,221,763, 5,212,324, 5,739,161 and 6,242,485, 7,253,295 and US publication No. 2006-0194880 (these cited references are herein incorporated by reference).

According to the present invention a mammalian subject may be treated by the instant invention by administering the compound used in the present invention. The subject may be any mammalian subject including a human. The compound may be applied systemically or topically. Usually, the compound may be administered by oral administration, intranasal administration, inhalational administration, intravenous injection (including infusion), subcutaneous injection, intra rectal administration, intra vaginal administration, transdermal administration and the like.

The dose may vary depending on the strain of the animal, age, body weight, symptom to be treated, desired therapeutic effect, administration route, term of treatment and the like. A satisfactory effect can be obtained by systemic administration 1-4 times per day or continuous administration at the amount of 0.00001-500 mg/kg per day, more preferably 0.0001-100 mg/kg.

The compound may preferably be formulated in a pharmaceutical composition suitable for administration in a conventional manner. The composition may be those suitable for oral administration, intranasal administration, inhalational administration, injection or perfusion as well as it may be an external agent, suppository or pessary.

The composition of the present invention may further contain physiologically acceptable additives. Said additives may include the ingredients used with the present compounds such as excipient, diluent, filler, resolvent, lubricant, adjuvant, binder, disintegrator, coating agent, cupsulating agent, ointment base, suppository base, aerozoling agent, emulsifier, dispersing agent, suspending agent, thickener, tonicity agent, buffering agent, soothing agent, preservative, antioxidant, corrigent, flavor, colorant, a functional material such as cyclodextrin and biodegradable polymer, stabilizer. The additives are well known to the art and may be selected from those described in general reference books of pharmaceutics.

The amount of the above-defined compound in the composition of the invention may vary depending on the formulation of the composition, and may generally be 0.000001-10.0%, more preferably 0.00001-5.0%, most preferably 0.0001-1%.

Examples of solid compositions for oral administration include tablets, troches, sublingual tablets, capsules, pills, powders, granules and the like. The solid composition may be prepared by mixing one or more active ingredients with at least one inactive diluent. The composition may further contain additives other than the inactive diluents, for example, a lubricant, a disintegrator and a stabilizer. Tablets and pills may be coated with an enteric or gastroenteric film, if necessary. They may be covered with two or more layers. They may also be adsorbed to a sustained release material, or microcapsulated. Additionally, the compositions may be capsulated by means of an easily degradable material such gelatin. They may be further dissolved in an appropriate solvent such as fatty acid or its mono, di or triglyceride to be a soft capsule. Sublingual tablet may be used in need of fast-acting property.

Examples of liquid compositions for oral administration include emulsions, solutions, suspensions, syrups and elixirs and the like. Said composition may further contain a conventionally used inactive diluents e.g. purified water or ethyl alcohol. The composition may contain additives other than the inactive diluents such as adjuvant e.g. wetting agents and suspending agents, sweeteners, flavors, fragrance and preservatives.

The composition of the present invention may be in the form of spraying composition, which contains one or more active ingredients and may be prepared according to a known method.

Example of the intranasal preparations may be aqueous or oily solutions, suspensions or emulsions comprising one or more active ingredient. For the administration of an active ingredient by inhalation, the composition of the present invention may be in the form of suspension, solution or emulsion which can provide aerosol or in the form of powder suitable for dry powder inhalation. The composition for inhalational administration may further comprise a conventionally used propellant.

Examples of the injectable compositions of the present invention for parenteral administration include sterile aqueous or non-aqueous solutions, suspensions and emulsions. Diluents for the aqueous solution or suspension may include, for example, distilled water for injection, physiological saline and Ringer's solution.

Non-aqueous diluents for solution and suspension may include, for example, propylene glycol, polyethylene glycol, vegetable oils such as olive oil, alcohols such as ethanol and polysorbate. The composition may further comprise additives such as preservatives, wetting agents, emulsifying agents, dispersing agents and the like. They may be sterilized by filtration through, e.g. a bacteria-retaining filter, compounding with a sterilizer, or by means of gas or radioisotope irradiation sterilization. The injectable composition may also be provided as a sterilized powder composition to be dissolved in a sterilized solvent for injection before use.

The present external agent includes all the external preparations used in the fields of dermatology and otolaryngology, which includes ointment, cream, lotion and spray.

Another form of the present invention is suppository or pessary, which may be prepared by mixing active ingredients into a conventional base such as cacao butter that softens at body temperature, and nonionic surfactants having suitable softening temperatures may be used to improve absorbability.

The term “treatment” or “treating” used herein includes any means of control such as prevention, care, relief of the condition, attenuation of the condition and arrest of progression.

According to the present invention, the prostaglandin compound protects mitochondria from various damage, and therefore, the prostaglandin compound is useful for the treatment of mitochondorial dysfunction/mitochondorial disease and a condition or disease associated with mitochondrial dysfunction, especially aging.

In the instant specification and claims, “condition or disease associated with mitochondrial dysfunction” includes any condition or disorder which is directly or indirectly caused by mitochondrial dysfunction and may include the diseases of brain, nerves, muscles, heart, eyes, kidneys, respiratory problems. The pharmaceutical composition of the present invention may further contain other pharmacological ingredients as far as they do not contradict the purpose of the present invention.

Further details of the present invention will follow with reference to test examples, which, however, are not intended to limit the present invention.

### EXAMPLE 1

### Methods

Human pulmonary Artery Smooth Muscle cells (PASMC) were grown to confluence on 10×22 mm glass cover slips in Clonetics smooth muscle basal media supplemented with growth hormones and fetal bovine serum. The cells on glass cover slips were then placed in 3 ml Hank's Balanced Salt Solution (HBSS) supplemented with 12 mM of the mitochondrial dye JC-1 and incubated at 37° C. for 30 minute. The cells on the cover slips were then washed with 3 ml HBSS and mounted in a cuvette in a spectrofluorimeter with 3 ml HBSS. The emission spectra with excitation at 490 nm were taken over the range of 520 nm to 620 nm (150 sec). 250M (0.1% acetone) FCCP, which leads complete depolarization of the mitochondrial membrane potential, was added at the end of each experiment. The spectra were normalized by dividing them by the fluorescence at 570 nm. The value obtained after FCCP treatment was assigned a value of 0 mV and the individual FCCP ratio was subtracted from each ratio point. The control (JC-1) fluorescence ratio was then assigned a value of +224 mV and the membrane potential [D_{mH}(mV)] for each experimental point were calculated accordingly.

### EXAMPLE 1-1

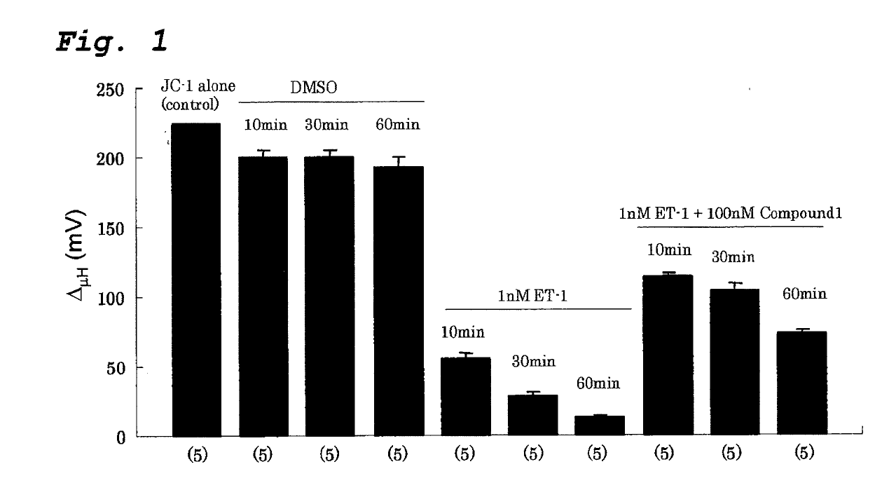

Effect of compound 1 (11-deoxy-13,14-dihydro-15-keto-16,16-difluoro-PGE₁) on Endothelin-1 (ET-1) induced loss of mitochondrial membrane potential of human pulmonary artery smooth muscle cells was examined (FIG. 1).

The cells were treated with 0.1% DMSO (vehicle for Compound 1), 1 nM Endothelin-1 (ET-1) or 1 nM ET-1 and 100 nM Compound 1 and scans were taken after 10, 30 and 60 minutes of the incubation. FCCP was added at the end and incubated for 60 minutes before taking scan. N=5 cover slips for all conditions.

### Result

The control mitochondrial membrane potential (JC-1) was taken to be 224 mV. When the cells were treated with 1 nM ET-1 for 10, 30 and 60 min, the mitochondrial membrane potential decreased to 54.5±4.3 mV, 28.0±2.8 mV, and 13.1±1.0 mV, respectively. The changes were all highly significant (p<0.001 in respect to control and DMSO). Compound 1 protects against ET-1 induced loss of membrane potential at all times tested. When the cells were treated with 1 nM ET-1 and 100 nM Compound 1 for 10, 30 and 60 min, the mitochondrial membrane potential was 114.2±2.6 mV, 104.02±5.0 mV, and 73.1±2.4 mV, respectively. These were significantly protected p<0.001, compared to the respective values from treatments with ET-1 alone.

### EXAMPLE 1-2

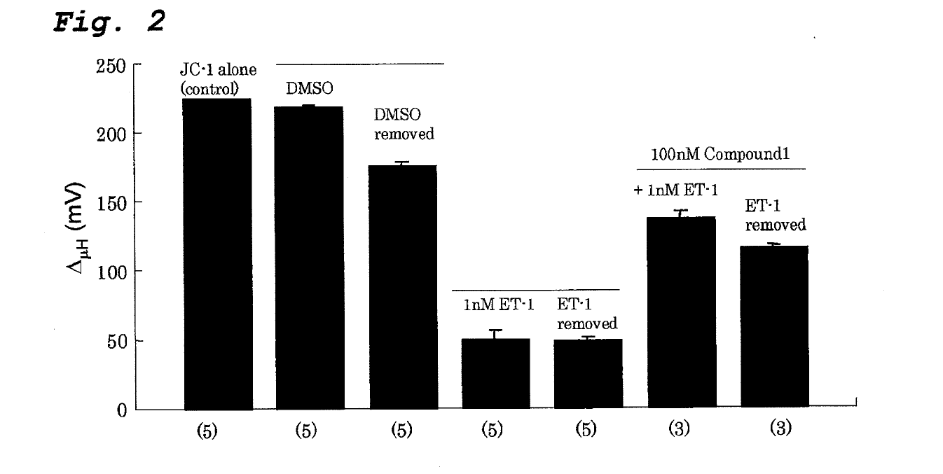

Effect of Compound 1 on ET-1 induced irreversible loss of mitochondrial membrane potential of human pulmonary artery smooth muscle cells (PASMC) was examined (FIG. 2).

The cells were treated with 0.1% DMSO or 1 nm Endothelin-1 (ET-1) and scans were taken after 30 minutes of incubation. The medium was then removed by washing the cells with fresh HBSS and scans were taken after 30 minutes. FCCP was added to the dish at the end and incubated for 60 minutes before taking scan. N=5 cover slips. In other set of 5 cover slips 1 nM Endothelin-1 (ET-1) and 100 nM Compound 1 were added and scans were taken after 30 minutes of incubation. The medium was then removed by washing the cells with fresh HBSS and 100 nM Compound 1 was added on top of it. Scans were taken after 30 minutes. FCCP was added at the end and incubated for 60 minutes before taking scan. N=3 cover slips.

### Result

There was a small effect of treatment 0.1% DMSO for 30 min to 217.8±1.0 mV which decreased further (176±2.8 mV) after DMSO was removed. Treatment with 1 nM ET-1 for 30 min caused a loss of mitochondrial membrane potential to 50.3±5.6 mV, which persisted even when ET-1 was subsequently removed (49.3±2.2 mV). Thus, ET-1 causes an irreversible loss of mitochondrial membrane potential. 1 nM ET-1 along with 100 nM Compound 1 reduced the mitochondrial membrane potential to 137.1±4.7 mV, and with subsequent removal of ET-1 with continued treatment with Compound 1 it was 115.3±2.3 mV. There was a significant protection by Compound 1 against a loss of membrane potential by ET-1. (FIG. 2)

### EXAMPLE 1-3

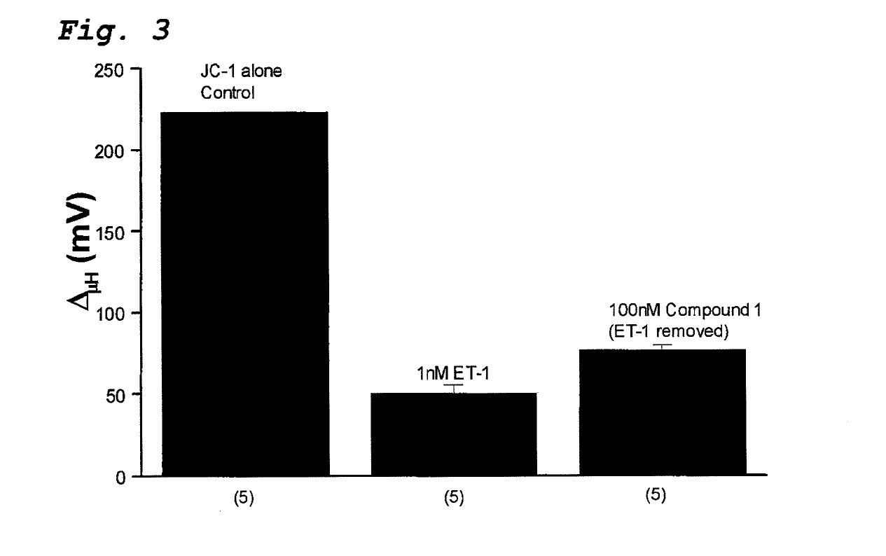

Rescue effect of Compound 1 on ET-1 induced loss of mitochondrial membrane potential was examined (FIG. 3).

The cells were treated with 1 nM Endothelin-1 (ET-1) and scans were taken after 30 minutes of incubation. The medium was then removed by washing the cells with fresh HBSS and HBSS supplemented with 100 nM Compound 1 was added to the cells. Scans were taken after 30 minutes. FCCP was added at the end and incubated for 60 minutes before taking scan. N=5 cover slips for all conditions.

### Result

As shown in FIG. 3, the loss of mitochondrial membrane potential to 50.3±5.6 mV caused by 30 minute treatment with 1M ET-1 was significantly (p<0.001) reversed by subsequent removal of ET-1 and inclusion of 100 nM Compound 1, and raised to 77.2±2.7 mV. Thus, Compound 1 not only protects against a loss in mitochondrial membrane potential as seen when Compound 1 is present throughout the time of treatment with ET-1 (FIGS. 1 and 2), but also can reverse the loss observed with ET-1.

The results show that Compound 1 protects mitochondria.

### EXAMPLE 2

### Methods

Smoke from 8 cigarettes were bubbled slowly through 100 ml of serum free culture medium and the resulting suspension was filtered through 0.20 μm filter. The solution was considered as 100% cigarette smoke extract (CSE). Human lung alveolar type II cells (A549) were seeded 1.5×10⁵ cells per well in a 96 well plate and incubated for 48 hrs. The cells were then treated separately with either 100 nM Compound A (13,14-dihydro-15-keto-16,16-difluoro-18(S)-methyl-PGE₁) or 1%, 2.5% and 5% CSE. In other sets, 100 nM Compound A was added along with 1%, 2.5% or 5% CSE. All incubations were done at 37° C. for 24 hrs. After 24 hr treatment, the cells were washed with 0-4° C. PBS three times. Measurement of cytochrome c that was translocated into the cytosol, a marker of cellular injury, was performed with a cytochrome c ELISA assay kit according to the instructions provided with the kit.

### Results

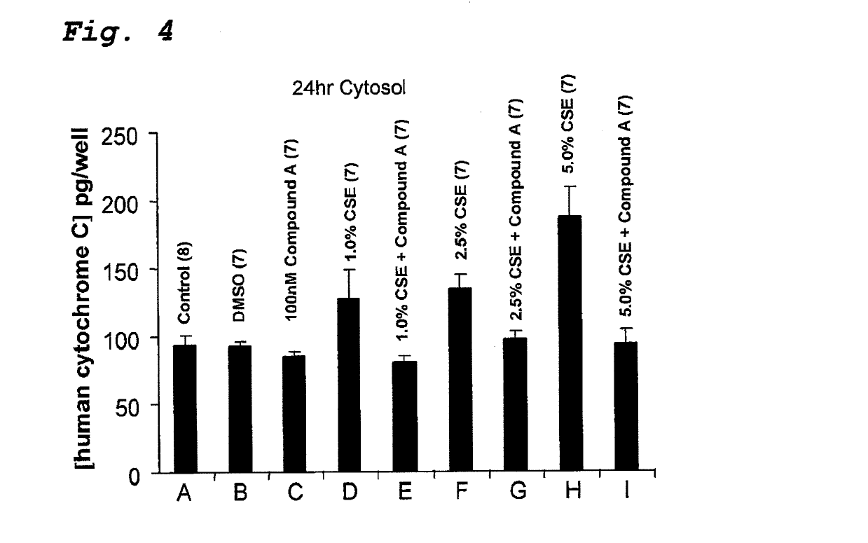

Results are summarized in FIG. 4. Cytochrome c translocation was measured. CSE caused a significant increase in cytochrome c translocation in a dose dependent manner. Neither 0.1% DMSO (B) (vehicle for Compound A) nor Compound A (C) significantly affected cytosolic cytochrome c. At all doses of CSE, 100 nM Compound A (E, G, I) protected against cytochrome c translocation by CSE. Data are expressed as mean ±SEM pg/well, n, the number of wells per point is shown above each bar. Data are expressed as pg/well cytochrome c.

The results demonstrate protective effects of Compound A on mitochondria of alveolar cells.

While the invention has been described in detail and with reference to specific embodiments thereof, it will be apparent to one skilled in the art that various changes and modifications can be made therein without departing from the spirit and scope thereof.

What is claimed is:

1. A method for the protection of mitochondria from damage in a mammalian subject, which comprises administering to the subject in need thereof an effective amount of a prostaglandin compound represented by the following general formula (I): 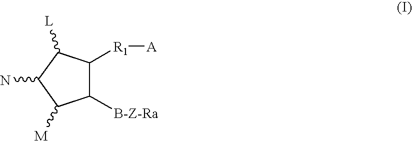
wherein L, M and N are hydrogen atom, hydroxy, halogen atom, lower alkyl, hydroxy(lower)alkyl, lower alkanoyloxy or oxo, wherein at least one of L and M is a group other than hydrogen, and the five-membered ring may have at least one double bond;
A is —CH₃, or —CH₂OH, —COCH₂OH, —COOH or a functional derivative thereof;
B is single bond, —CH₂—CH₂—, —CH_CH—, —C≡C—, —CH₂—CH₂—CH₂—, —CH═CH—CH₂—, —CH₂—CH═CH—, —C≡C—CH₂— or —CH₂—C≡C—;
Z is 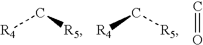
or single bond
wherein R₄ and R₅ are hydrogen, hydroxy, halogen, lower alkyl, lower alkoxy or hydroxy(lower)alkyl, wherein R₄ and R₅ are not hydroxy and lower alkoxy at the same time;
R₁ is a saturated or unsaturated bivalent lower or medium aliphatic hydrocarbon residue, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur; and
Ra is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group.

2. The method as described in claim 1, wherein said prostaglandin compound is 16-mono or dihalogen-prostaglandin compound.

3. The method as described in claim 1, wherein said prostaglandin compound is 15-keto-prostaglandin compound.

4. The method as described in claim 1, wherein said prostaglandin compound is 13,14-dihydro-16-mono or dihalogen-prostaglandin compound.

5. The method as described in claim 1, wherein said prostaglandin compound is 13,14-dihydro-15-keto-prostaglandin compound.

6. The method as described in claim 1, wherein said prostaglandin compound is 13,14-dihydro-15-keto-16-mono or dihalogen-prostaglandin compound.

7. The method as described in claim 1, wherein said prostaglandin compound is 13,14-dihydro-16-mono or difluoro-prostaglandin compound.

8. The method as described in claim 1, wherein said prostaglandin compound is 15-keto-16-mono or difluoro-prostaglandin compound.

9. The method as described in claim 1, wherein said prostaglandin compound is 13,14-dihydro-15-keto-16-mono or difluoro-prostaglandin compound.

10. The method as described in claim 1, wherein said prostaglandin compound is 13,14-dihydro-16-mono or dihalogen-prostaglandin E compound.

11. The method as described in claim 1, wherein said prostaglandin compound is 15-keto-16-mono or dihalogen-prostaglandin E compound.

12. The method as described in claim 1, wherein said prostaglandin compound is 13,14-dihydro-15-keto-16-mono or dihalogen-prostaglandin E compound.

13. The method as described in claim 1, wherein said prostaglandin compound is 13,14-dihydro-16,16-difluoro-prostaglandin E₁ compound.

14. The method as described in claim 1, wherein said prostaglandin compound is 13,14-dihydro-15-keto-prostaglandin E₁ compound.

15. The method as described in claim 1, wherein said prostaglandin compound is 11-deoxy-13,14-dihydro-15-keto-16,16-difluoro-prostaglandin E₁ compound or 13,14-dihydro-15-keto-16,16-difluoro-18-methyl-prostaglandin E₁ compound.

16. A method for treating a mitochondrial dysfunction in a mammalian subject, which comprises administering to the subject in need thereof an effective amount of a prostaglandin compound represented by the following general formula (I): 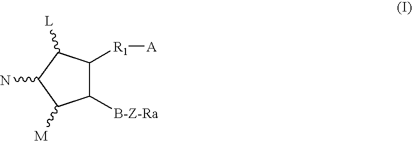
wherein L, M and N are hydrogen atom, hydroxy, halogen atom, lower alkyl, hydroxy(lower)alkyl, lower alkanoyloxy or oxo, wherein at least one of L and M is a group other than hydrogen, and the five-membered ring may have at least one double bond;
A is —CH₃, or —CH₂OH, —COCH₂OH, —COOH or a functional derivative thereof;
B is single bond, —CH₂—CH₂—, —CH═CH—, —C≡C—, —CH₂—CH₂—CH₂—, —CH═CH—CH₂—, —CH₂—CH—CH—, —C≡C—CH₂— or —CH₂—C≡C—;
Z is 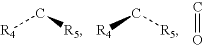
or single bond
wherein R₄ and R₅ are hydrogen, hydroxy, halogen, lower alkyl, lower alkoxy or hydroxy(lower)alkyl, wherein R₄ and R₅ are not hydroxy and lower alkoxy at the same time;
R₁ is a saturated or unsaturated bivalent lower or medium aliphatic hydrocarbon residue, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur; and
Ra is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy;
cyclo(lower) alkyl; cyclo(lower)alkyloxy; aryl; aryloxy; heterocyclic group; heterocyclic-oxy group.

17. A method for treating a condition associated with mitochondrial dysfunction in a mammalian subject, which comprises administering to the subject in need thereof an effective amount of a prostaglandin compound represented by the following general formula (I): 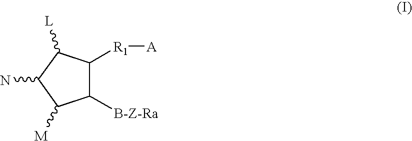
wherein L, M and N are hydrogen atom, hydroxy, halogen atom, lower alkyl, hydroxy(lower)alkyl, lower alkanoyloxy or oxo, wherein at least one of L and M is a group other than hydrogen, and the five-membered ring may have at least one double bond;
A is —CH₃, or —CH₂OH, —COCH₂OH, —COOH or a functional derivative thereof;
B is single bond, —CH₂—CH₂—, —CH═CH—, —C≡C—, CH₂—CH₂—CH₂—, —CH═CH—CH₂—, —CH₂—CH═CH—, —C≡C—CH₂— or —CH₂—C≡C—;
Z is 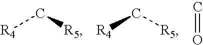
or single bond
wherein R₄ and R₅ are hydrogen, hydroxy, halogen, lower alkyl, lower alkoxy or hydroxy(lower)alkyl, wherein R₄ and R₅ are not hydroxy and lower alkoxy at the same time;
R₁ is a saturated or unsaturated bivalent lower or medium aliphatic hydrocarbon residue, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur; and
Ra is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower) alkyl; cyclo(lower) alkyloxy; aryl; aryloxy; heterocyclic group; heterocyclic-oxy group.
